(19) United States
(12) Patent Application Publication
(10) Pub. No.: US 2025/0114036 A1
(43) Pub. Date: Apr. 10, 2025

(54) METHOD FOR ANALYZING USEFUL DATA OF AN ORTHOPEDIC DEVICE

(71) Applicant: Otto Bock Healthcare Products GmbH, Vienna (AT)

(72) Inventor: Dirk Seifert, Vienna (AT)

(21) Appl. No.: 18/256,331

(22) PCT Filed: Dec. 7, 2021
(86) PCT No.: PCT/EP2021/084491

§ 371 Date: Jun. 7, 2023

(30) Foreign Application Priority Data
Dec. 8, 2020 (DE) 102020132551.7

(51) Int. Cl. A61B 5/00; A61F 2/64; A61F 2/76; A61F 5/01; G16H 20/40; G16H 40/67

(52) U.S. Cl. CPC A61B 5/4851; A61B 5/6811; A61B 5/6812; A61F 2/64; A61F 5/0123; G16H 20/40; G16H 40/67; A61F 2002/7615; A61F 2005/0188

(57) ABSTRACT

The invention relates to a method for analyzing useful data of an orthopedic device, having the steps of: providing an orthopedic device (10, 13) which has at least one sensor (30) for detecting sensor data and/or which is connected to a data preparing device (70) for transmitting sensor data, comprising at least one data processing device (40) which is coupled to the at least one sensor (30) and/or the data preparing device (70) and which is equipped with at least one storage device for storage purposes and a processor for processing the sensor data; ascertaining characteristic variables and/or characteristic variable combinations; detecting an event or a time-limited activity using the sensor data ascertained at a point in time or in a period of time and/or using a sensor data combination; ascertaining characteristic variables of the orthopedic device (10, 13) via the at least one sensor (30) and/or the data preparing device (70); assigning each characteristic variable and/or characteristic variable combination to an event or a time-limited activity; and storing the occurrence of the event or the time-limited activity together with at least one characteristic variable or characteristic variable combination.

The invention relates to a method for analyzing usage data of an orthopedic device. Orthopedic devices are in particular prostheses, orthoses, exoskeletons, wheelchairs, bandages, and other devices which can be used as a replacement for or to assist physical functions. In particular, orthoses and prostheses and exoskeletons are viewed as orthopedic devices.

It is helpful and sometimes necessary to obtain comprehensive information about the use of orthopedic devices in both the development and the use of orthopedic devices. The information which is required is varying and can relate to many fields of the loads and uses of the orthopedic device. For example, mechanical and thermal loads during the use of the orthopedic device can be of interest, for example to check dimensioning, determine service lives, or recognize or predict disturbances in the functional sequence. It is also possible and helpful to determine movement profiles or usage profiles in order to recognize in which areas and in which context the orthopedic device is used. Functionalities and dimensioning may thus be adapted to the actual application. It is also possible to make it visible to the user himself how the assistive device is used and to display the use of the assistive device, in particular via the representation of the usage data in an app, for example on a mobile terminal such as a smart phone or a tablet.

Prosthetic devices are already known from the prior art, using which individual events are detected and stored. In the prosthetic knee joint “C-Leg”*® from Ottobock, steps on the level and ramp steps are logged via a step counter. Upon each occurrence, the numeric value is increased by a value. Furthermore, a monitoring function exists in this prosthetic knee joint of the activities in which the number of the steps is counted in different speed classes and with specific loads. Various situations are detected by means of a time function, for example the time consumed in an auxiliary mode or while standing. The criteria for incrementing in the counter or adding times in the time function are defined beforehand, only the increasing number of the events and the duration of the states are counted. If errors occur during the use of the prosthetic knee joint, the respective errors are provided with an error code and stored in an error memory.

In research programs, so-called data loggers are used for a more detailed detection of data, which are designed as an external device and continuously record digital and analog signals. The signals are sensor signals, so that signals and states can be completely recorded over multiple days via data loggers. This results in very large amounts of data which have to be analyzed over the entire period of time thereafter. The circumstance that sensors and a data logger are used together with the orthopedic device obstructs the detection of data and the possibility of obtaining realistic data about a use under realistic conditions. In addition, the very large amounts of data have to be complexly processed and analyzed.

The object of the present invention is to provide a method which provides reliable, realistic, and detailed information about the use of orthopedic devices, has a low storage requirement and analysis expenditure, and enables the amount of data possibly to be transmitted to other devices to be reduced.

This object is achieved according to the invention by a method having the features of the main claim. Advantageous embodiments and refinements of the invention are disclosed in the dependent claims, the description, and the figures.

The method for analyzing usage data of an orthopedic device provides providing an orthopedic device, which includes at least one sensor for detecting sensor data and/or is connected to a data provision device for transmitting sensor data. The orthopedic sensor device includes a data processing device, which is coupled to the at least one sensor and/or the data provision device, wherein at least one memory for storing and one processor for processing the sensor data are present in the data processing device. Other data, in particular processed or analyzed data, are also viewed as sensor data, for example position data from a GPS system, weather data, statistical data, or other data which are determined, measured, or provided, processed or analyzed and transmitted via the data provision device in any manner.

Moreover, characteristic parameters and/or parameter combinations are defined. An event or a time-limited activity is recognized on the basis of sensor data and/or sensor data combinations determined at a point in time or in a period of time. This is carried out, for example, by the analysis of sensor data during the use of the orthopedic device. Alternatively or additionally, data from a data provision device can also be used to recognize an event or a time-limited activity.

After an event or a time-limited activity is recognized, the associated characteristic parameters or characteristic parameter combinations are associated with the respective event or the respective time-limited activity. The characteristic parameters are determined in particular during the use of the orthopedic device via the sensors and/or the data provision device. The characteristic parameters and/or parameter combinations are associated with the event or the time-limited activity and thus qualified. The occurrence of the event or the time-limited activity having at least one characteristic parameter or parameter combination is stored, either in the orthopedic device or in another data processing device, possibly after processing of the data.

Events are recognized by the data processing device on the basis of the detected and/or transmitted sensor data and criteria stored in the memory, wherein an event is to be understood not only as a moment in time, but also a time-limited activity. At least one characteristic parameter is associated with at least one of the events, which is determined at the runtime from at least one detected and/or transmitted parameter and/or its time curves and is stored together with information about the occurrence of the event in the memory. It is thus possible to store both the event and the parameter characteristic for the event, in particular the combination of the two.

Instead of the complete storage of data streams over a long period of time, it is possible using the claimed method to reduce the amount of data and only still detect 78 the events or the time-limited activity and the associated characteristic parameters, for example by counting the events.

Both the event as such and also parameters typical for the event or defining the event can be stored. Instead of a continuous recording of raw data, only a fraction of the computing power and storage space is required to analyze the data or transmit the data to other devices. In comparison to solely counting predefined events, the characteristic parameters stored together with the events offer a significantly higher information content. A subsequent analysis is significantly simplified, since an association of characteristic parameters with the events or the time-limited activities and their analysis or calculation has already taken place during the use of the orthopedic device or during the runtime. In addition to the sensors, which are arranged directly on the orthopedic device or can be assigned thereto, sensor data, parameters, or other information can be transmitted via one data provision device or multiple data provision devices. These sensor data, parameters, or information are, for example, internal states of the orthopedic device or data from data memories, a connection to another device in a network, the Internet of Things (IoT), the Internet or the cloud, satellites, space probes, from other orthopedic devices, from electronic components, so-called wearables such as smart watches, fitness armbands, mobile telephones, or the like.

The sensor data or parameters of the orthopedic device are determined in one embodiment by analyzing the sensor data and/or those information which are transmitted from the data provision device, by way of the processor, which can be part of a control device. For this purpose, the sensor values can in particular be amplified, filtered, or processed in another manner. It is also possible to combine multiple sensor values and/or values of the data provision device with one another.

An event or a predefined time-limited activity is recognized from a comparison to previously defined sensor data or parameters and/or sensor data combinations or parameter combinations on the basis of the criteria stored in the memory and classified as such, possibly together with the time information. The criteria can involve a comparison of sensor data to one or more limiting values. However, it is also possible that sensor data combinations and/or their time curves are used for recognizing events, for example by linking multiple sensor data by arithmetic operations, calculation rules, algorithms and/or by processing multiple parameters or their time curves in one or more classifiers combined with one another. Classifiers are, for example, decision trees, state machines, artificial neural networks (ANN), recurrent and/or convolutional neural networks, linear discrimination, support vector machines, hidden Markov models, deep neural networks, or similar methods for classifying data. Time-limited activities can be recognized, inter alia, by the recognition of a starting event and an end event.

Events can be, for example, interactions of the user with the orthopedic device, such as the connection to peripheral devices, for example a smart phone, a tablet, a computer, a smart watch, or a fitness watch. These also include the connection to a charging device or the coupling to another orthopedic device and/or components of orthopedic devices, the actuation via interfaces, such as for example software, buttons, joysticks, and/or other operating elements. Interactions via movement patterns are also included here, for example switching between operating modes by way of special movements. Events can be defined solely chronologically, for example hourly, daily, and/or weekly events. Spatial conditions can also result in events, for example entering or leaving a spatially delimited area, covering a distance, exceeding or falling below velocities and/or the spatial distance to another device and/or a person and their time curve, for example the duration of a small distance to this and/or that. Furthermore, it is possible that internal states of the orthopedic device are defined as events, such as for example system errors, reaching critical system states and/or other internal states or state changes which are of interest. Physical events in the scope of the use of the orthopedic device can also be defined as events. These include in particular putting on and/or taking off the orthopedic device, the occurrence of defined events in movement sequences, such as for example the initial contact, the initiation of a swing phase, and/or the toe-off in prostheses and/or orthoses of the lower extremity. In the case of a hand prosthesis or hand orthosis, this includes, for example, switching between grip types, assuming a grip type, and/or opening and/or closing a grip. Assuming specific positions also counts as an event, for example being seated or sitting down, standing up and/or lying down and/or the occurrence of a load and/or relief, sudden load change, shocks, falls, and/or temperature changes. It is also possible that an event is triggered via a data provision device, for example via a connection to another device, a network, the Internet, and/or the cloud and thus by external data, which are made available to the orthopedic device, or corresponding commands transmitted to the orthopedic device.

In particular activities which are delimited by the described, generally singular events are viewed as time-limited activities. These are in particular physical activities which the user carries out using the orthopedic device, for example executing a step, sitting down, holding an object, walking down a staircase, remaining in a position, and/or bicycling. However, it is also possible that the interaction via other devices or operating elements represents a time-limited activity, for example the existing connection to another device, for example to a smart phone, the charging via a charging device, or the period of time between two interactions, for example two changeovers of the operating mode by the user.

One refinement of the invention provides that the occurrence of a respective event or the appearance of the time-limited activity is stored with an associated time information or a plurality of time information, in particular with at least one unique timestamp. The characteristic parameter, using which the event or the time-limited activity is qualified, is thus time information or is provided with time information. In the case of a time-limited activity, it is advantageous in particular to store time information in each case for the beginning and the end of the activity. It is also possible to store the duration of a limited activity, in particular together with a further timestamp. To further reduce the amount of data, it is moreover possible to store a complete timestamp only for certain points in time and/or events and in between to use relative timestamps, which have a lesser data length.

One refinement of the invention provides that additionally or alternatively to the time information, other characteristic parameters are stored, which are related to the respective associated events and describe their qualities, in particular characteristic parameters which are determined at the run time from multiple sensor values and/or values of the data provision device and/or from the time curve of at least one parameter. By way of such characteristic parameters, an event can be described by a few values already calculated during the run time, by which the storage requirement is significantly reduced. Such characteristic parameters can be parameters, for example, which describe a movement sequence. In the case of a leg prosthesis, this can be, for example, the duration, length, or height difference of a step, the maximum knee angle reached in the swing phase, or the extent of stance phase bending or plantar flexion in the stance phase. The parameters can relate to both the movement carried out by the user and also to movements of the orthopedic device itself. It is also possible that the at least one characteristic parameter describes a load of the orthopedic device to be associated with the event or the load of the user. The load is, for example, a force or a torque, respectively, applied to the orthopedic device and/or acting in the orthopedic device, in particular a maximum or average force or torque. Alternatively or additionally, the load is the force applied by the user or acting on the user or the applied or acting torque. However, work performed and/or a performance can also be used as a characteristic parameter, as can a temperature or a power consumption as a result of a load. For example, the gripping force, for example of a hand prosthesis, a ground reaction force, a force engagement point, in particular the center of pressure (CoP) on the foot, a knee torque, or the forces acting on a body part, for example a prosthesis or orthosis of the lower extremity, can be used. In a prosthesis or orthosis of the lower extremity, for example, the work performed in a step or a stepping phase by a resistance device and/or an actuator can be stored as a characteristic parameter.

In particular in combination with sensors and/or external devices which detect biosignals, these can be stored as characteristic parameters. For example, the pulse, the breath rate, and/or a variability of the heart rate of the user can be detected by the orthopedic device itself or, for example, a pulse watch or a smart watch and stored as a characteristic parameter.

In the case of an interaction with an external device, information about the participating components, details on the connection, and/or the type of the interactions themselves can be used as characteristic parameters. It is thus possible, for example, that upon the connection of the orthopedic device to a smart phone via a radio connection, an ID is stored which identifies the smart phone, such as for example the MAC address or the IMEI. Upon such a connection, it is also possible to store which software, app, or the versions thereof were used to communicate with the orthopedic device. Upon the connection to a charging device, the state of charge of the internal energy store of the orthopedic device can be used as a characteristic parameter.

Spatial or position information, such as for example the position of the orthopedic device both absolutely in the meaning of geographic data and also in relation to other objects or subjects, for example the presence in a room and/or vehicle and/or the proximity to an external device and/or person is also suitable as a characteristic parameter. These data can be made available both by sensors of the orthopedic device and also via the data provision device. Furthermore, information about the orientation in space, the velocity, and/or accelerations can be stored as characteristic parameters. In particular, characteristic parameters can be used which give information about the quality of a movement carried out or the coordination of the user. These are, for example, the variance of the velocity or the orientation, the precision of a gripping or positioning movement in terms of a uniform and/or the most direct possible movement and/or the progress of the body center of gravity or the center of pressure during a step.

Environmental parameters can also be used as characteristic parameters, in particular the ambient temperature, the ambient humidity, or the quality of objects with which the orthopedic device interacts, for example the quality of the underlying surface, in particular the inclination of the underlying surface. Thus, for example, the size, the shape, or the weight of an object which is gripped by a hand prosthesis or orthosis can be determined at the run time and stored as a characteristic parameter. It is also possible to determine and store the inclination of the underlying surface during a step. Furthermore, internal variables of the orthopedic device can be used as characteristic parameters, such as for example control variables or parameters which describe the program sequence in the controller of the orthopedic device. In particular, it is possible in the event of system errors to associate these values as characteristic parameters with the error and store them. Furthermore, characteristic parameters can be defined which describe the time curve of a parameter, a trajectory, or geometric arrangement by way of a parametric representation. This is, for example, the description via a Fourier series, shapelets, polynomials, splines, or basis functions.

The storage can be performed within the orthopedic device, wherein the raw amount of data no longer has to be stored, rather only the occurrence of the at least one event and one or more possible associated characteristic parameters, in particular time information. For efficient storage of the events, these can be assigned identifiers, so-called LDs. An ID represents a specific event, wherein the meaning of the ID does not necessarily also have to be stored. In relation to storing all determined data, the amount of data to be stored and possibly to be processed and/or transmitted later is reduced to a fraction, wherein a reduction factor of 200 to 500 can be assumed. This moreover reduces the energy consumption of the processor and the storage device.

In relation to solely storing the number of events which has occurred, via the possibly associated characteristic parameters, significant features of the invents and thus the use are stored and are available for further analysis. In particular if the time information is stored as one of the characteristic parameters, the time sequence and the time distribution of the events are stored. It can thus be determined during the use of a prosthetic knee joint while walking on the level that a step has been carried out with a stance phase flexion. It is possible in this case that in addition to storing the occurrence of this event, a timestamp and/or other parameters characteristic for the step are stored, for example the maximum knee angle reached in the stance phase flexion and/or the maximum load of the prosthesis in the stance phase. The characteristic parameters associated with the event are stored here in the memory, for example as a program, algorithm, or computing rule, and can be determined in real time or at the run time. At the same time, it can be categorized from the same parameters or transmitted information how many steps have been executed in a predetermined velocity class. The storage then takes place solely as a numeric variable in conjunction with time information within the respective category or association, without all raw data about the movement sequence while walking having to be stored and subsequently analyzed.

One refinement of the invention provides that the data stored at the run time are further processed at a later point in time by the orthopedic device, in particular upon lower computing load, upon the presence of an external power supply, and/or triggered by a corresponding interface, for example an upcoming data transmission to an external device. Further processing can be here in particular the calculation of statistics, the counting of events, in particular events which have specific characteristic parameters, the calculation of accumulative variables from the characteristic parameters, and/or methods for data compression. Such a later further processing has the advantage that computing-intensive and/or energy-intensive data processing processes take place at points in time in which sufficient computing power and energy are available and the energy consumption can be kept minimal at the runtime. Furthermore, accumulative variables, such as for example the number of steps per day using a prosthetic foot, can be calculated once, instead of having to update this number upon each step.

The events or time-limited activities stored over a period of time are advantageously analyzed to determine loads over a usage period of time, in particular mechanical and thermal loads and the reduction of performance features. The events stored over the course of a day, for example, are analyzed, for example when the orthopedic device is no longer being used, which can be recognized, for example, by the connection to a charging device. The analysis can take place within the orthopedic device or in an external device which is connected to the orthopedic device or to which the events or time-limited activities are transmitted. The transmission can take place via a wired connection or wirelessly, for example via a radio connection or also via the Internet.

The method can be used here for targeted studies within a restricted timeframe, so that an analysis mode is deliberately activated or a corresponding hardware component is connected to the required software devices over a certain period of time.

One refinement provides that the occurrence of the respective event or the time-limited activity is stored with the associated characteristic parameter and/or parameter combination and/or the associated time information. Due to the coupling of the event or the time-limited activity with the information about the characteristic parameter and/or the time information, an easier association and a more accurate analysis of the data can take place.

At least one time information can be associated with the at least one event or the time-limited activity, for example the duration of a load, a charging process, a movement, or the like. Information is thus appended to the event without data having to be stored over a long period of time. A single value is sufficient, with which the time information is then linked.

Multiple different sensors are advantageously used to detect the sensor data, in order to detect as much different sensor data as possible and to be able to generate as many characteristic parameters or parameter combinations as possible therefrom. The at least one sensor or the sensors can be selected, for example, from a group of sensors which comprises force sensors, torque sensors, angle sensors, position sensors, velocity sensors, acceleration sensors, spatial location sensors, inertial measuring units (IMU), time measuring units, temperature sensors, pressure sensors, optical sensors, in particular for detecting absorption, emission, and reflection, acoustic sensors, piezoelectric sensors, sensors for detecting biometric data such as fingerprint or retina, sensors for detecting the electromagnetic fields, sensors for detecting biosignals such as pulse, blood pressure, variance, chemical composition, in particular of blood, saliva, sweat, and other bodily fluids, muscle activation, nerve signals, bioimpedance, conductivity, voltmeter, lead electrodes, radar, ultrasonic, lidar, and sonar sensors. The sensors from the group of virtual sensors can also be used, and multiple sensors can be connected to one another, for example in the form of sensor fusion. On the basis of the sensor data, possibly in conjunction with further information or data from external data provision devices, during the use of the orthopedic device, a variety of studies is performed during the use of the orthopedic device. The characteristic parameters or parameter combinations are detected, for example, from states, continuous values, maximum values, mean values, time intervals, integrative variables, or timestamps. Information with respect to the identity and quality of the individual components of an orthopedic device can also be accepted, so that, for example, after an exchange of components, it may be established whether a change of the load or the usage behavior has occurred.

At least one sensor data range and/or one sensor data limiting value or at least one parameter range and/or parameter limiting value, reaching and/or exceeding or falling below which is defined as a condition for the occurrence of an event or as a definition of a time-limited activity, can be defined as sensor data or sensor data combinations or data or data combinations in general, which can trigger an event or which are used as a definition for an event or which define a time-limited activity. It is also possible that discrete values, continuous values, and time variables are used as sensor data or parameters, wherein the discrete values can also be designed as categories or values for categories. A category can be, for example, a color assignment, an assignment to a specific component category such as damper device, spring element, or structural component, a force type, or an operating mode, or the like.

The method is used in particular in prosthetic joints or orthotic joints and is used to analyze and check as well as optimize the orthosis or prosthesis during the use. In addition, instructions for improved use, improved maintenance, for reaching load limits, or about incorrect settings or the flawed use of technical components can be transmitted to the user on the basis of the analysis of usage data. The type of use, the duration of the use under certain conditions, and/or reaching a limiting value, in particular a load limit, can be determined on the basis of the number and/or time distribution of the respective stored events or the time-limited activities. A damage accumulation can be established and a precautionary request to carry out maintenance can be prepared. Load limits of the body of the user can also be recognized and made accessible to the respective user, a physician, or orthopedic technician.

The events or time-limited activities are advantageously analyzed and stored in the control device. Alternatively, the events or time-limited activities can be stored in the control device or the memory and transmitted at a later point in time to an external analysis device for analysis. It is also possible to transmit the time-limited activities and events after their identification to an external analysis device for analysis, for example by a wireless transmission via radio or another transmission device.

The storage of the events or the time-limited activities can be deactivatable, so that an analysis of the sensor data and/or the other information which are transmitted by the data provision devices only takes place when this is desired. In addition to a permanent analysis, the limiting to selected periods of time can be advisable with respect to the storage requirement and the data processing, in order to preserve resources and only analyze those data or situations which are helpful for answering predetermined questions. The storage can also be dependent on the existing operating mode, for example, if a state is detected which is not of interest for an analysis of the usage data, for example sitting, storage can be omitted for this state. The storage is only activated and the analysis is continued when this state changes, for example after standing up. The processing of the parameters and the analysis and storage can also be made dependent on the state of charge of the energy store. So as not to endanger the functionality of the orthopedic device, the data processing and storage and analysis can be omitted in order to minimize the energy consumption. If the state of charge of the energy store falls below a certain level, the storage and analysis is interrupted.

The definition of characteristic parameters and/or characteristic parameter combinations and/or the association of the respective characteristic parameters and/or parameter combinations with an event or the events can be changed, in particular parameters can be changed, on the basis of which the characteristic parameters and/or parameter combinations are defined. Both the quantity and the quality of the characteristic parameters and/or parameter combinations are changeable for one or more events or time-restricted activities. A parameter or at least one characteristic parameter or parameter combination can also be added or removed to the definition of events or time-limited activities. The parameters as such can also be exchanged or changed or the groups from which the parameter combinations are compiled can also change or can be changed. In particular, it is possible that the number and/or the type of the characteristic parameters and/or events is changed by an external device, such as a smart phone, a tablet, or a computer, which is connected to the orthopedic device.

The processing of sensor data, parameters, or events and/or time-limited activities provided with characteristic parameters or an analysis thereof advantageously takes place at predetermined times or in predetermined situations of the use of the orthopedic device. The processing or analysis of data advantageously takes place in times of low computing load, for example when the control device is not presently occupied with the processing of data for the usage operation of the orthopedic device. This can take place, for example, in the standby mode, during charging, when seated, and/or when lying down. It is also possible that the processing and/or transmission takes place when a connection to a network having correspondingly high transmission bandwidth takes place. This can be the case, for example, when the orthopedic device is coupled to an external transmission device, which is connected to a network or the Internet. In the case of a wireless transmission, the spatial distance between the external transmission device and the orthopedic device can be sufficient to establish a connection between them automatically and to carry out the processing and/or the transmission of the data.

The data storage can take place in a buffer memory having a low energy consumption, at least the energy consumption is to be less than in the memory of the control device, which is used for data processing to maintain the actual function of the orthopedic device. The sensor data, the external information of the data provision device, the characteristic parameters, the characteristic parameter combinations, and also the events or the time-limited activities with the characteristic parameters can be stored in the buffer memory. The data can be transmitted from the buffer memory regularly or in suitable situations, for example during inactivity, to a main memory, which can process comparatively large amounts of data. The overall energy consumption is thus reduced. So-called queries can be processed within the orthopedic devices, so that information is preprocessed within the orthopedic device and are only transmitted or further processed after the preprocessing. The queries can be created by external devices, sent to the orthopedic device and processed thereby. The results of such queries can as a consequence be transmitted to the external device.

Using the claimed method, it is possible to predefine, recognize, and store events or time-limited activities. Together with the event, characteristic parameters on this event are stored, for example time information. The event or the time-limited activity can be provided with a timestamp and stored and analyzed. In this case, a data analysis is carried out during the use of the orthopedic device and sensor raw data are combined to form specific clusters or events. Over the use of the orthopedic device, the corresponding events or clusters are then identified, qualified, and possibly provided with a timestamp and stored, for example saved in a table or database. The sequence, the number of the entries, the length, and the meaning of the individual entries can be freely defined and freely selected. The entries are stored as numeric values in succession in the form of tuples and/or as key value pairs or stored in other database structures. Instead of storing a large number of sensor values over the entire usage period of time and analyzing them after the usage, it is sufficient to monitor the sensor data over a certain period of time, recognize the corresponding events or time-limited activities, and provide them with previously defined characteristic parameters or parameter groups. If an event or a time-limited activity is recognized on the basis of the sensor data or criteria, a numeric value for this event or the time-limited activity is stored in the corresponding data field, possibly with a timestamp. This significantly reduces the amount of data and facilitates the analysis, without there being an information loss. All required information is present, which have been defined beforehand for events or time-limited activities to be studied.

Exemplary embodiments of the invention are explained in more detail hereinafter on the basis of the appended figures. In the figures:

FIG. 1—shows an orthopedic device in the form of an orthosis;

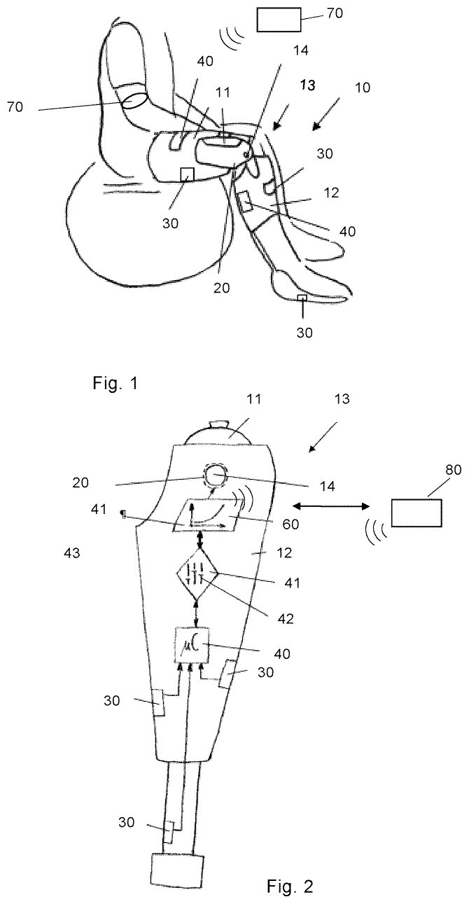

FIG. 2—shows a schematic representation of a prosthetic knee joint; and

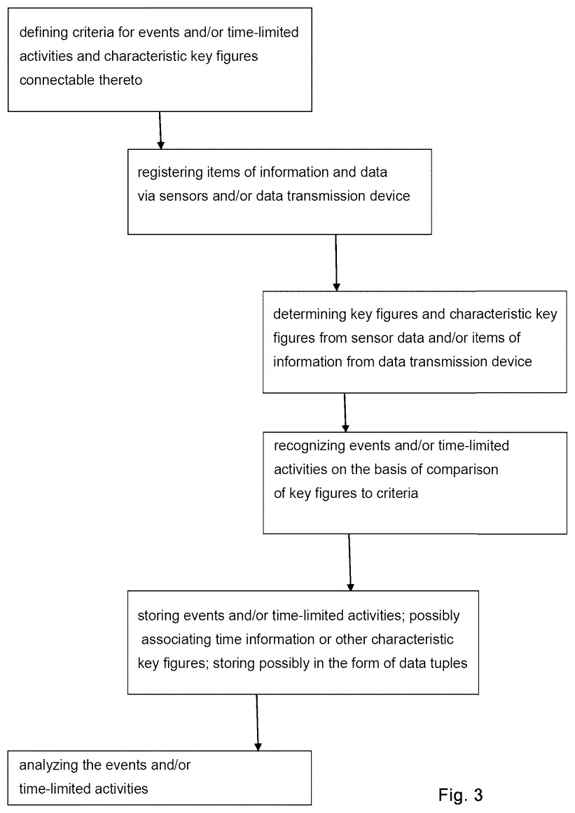

FIG. 3—shows a schematic representation of the method sequence.

FIG. 1 shows a schematic representation of an orthopedic device in the form of an orthosis 10 having an upper part 11, which is connected via a joint 13 to a lower part 12. The lower part 12 is connected around a pivot axis 14 to the upper part 11. The upper part 11 of the leg orthosis is designed as a shell or rail for accommodating a thigh, the lower part 12 is designed for contact on a lower leg and includes a foot part, which is formed on the lower part 12 or alternatively fastened thereon. A resistance device 20 is arranged on the upper part 11, via which a pivot resistance of the lower part 12 relative to the upper part 11 can be adjusted. The resistance device 20 can also be used in particular to block a pivot movement and is designed, for example, as a hydraulic damper, a brake, a magnetorheological resistance device, or the like. Sensors 30 are arranged on both the upper part 11 and the lower part 12, which can record information about the present spatial location, the position relative to one another, and possibly loads, angular positions, and/or accelerations, temperatures, acting forces, and other parameters of the respective components. The sensors 30 are coupled to a control device 40, either via cable or wirelessly. Within the control device 40, the sensor data and possibly other data and information from data transmission devices are analyzed in a data processing device and compared, for example, to limiting values or sensor data curves, which are stored in programs or memories within the control device 40. Control programs are stored in the control device 40, which influence the resistance device 20, for example increase or reduce a damping, while the user of the orthosis is walking. If the corresponding criteria for blocking or unblocking the joint 13 are met, the control device 40 outputs a control command to an adjustment device, which is arranged on the resistance device 20. The adjustment device, for example a motor or actuator for adjusting a valve, a coil for changing a magnetic field, or another actuating device or another influencing element, then acts within the resistance device 20 or on the resistance device 20 and blocks or unblocks the joint 13. Data processing programs are also stored in the control device 40, so that the control device 40 also assumes the data processing and analysis of the usage data.

During the use of the orthosis 10, the relevant sensor data or parameters are detected by the sensors 30 and/or provided by the data provision device 70 and transmitted to the control device 40. Criteria for specific events and/or time-limited activities are stored in the control device 40. These criteria are previously defined sensor data, sensor data curves, or sensor data combinations, wherein additional information, data, or sensor data can likewise be transmitted from the external data provision device 70 to the control device 40 and used as criteria. The information detected during the use of the orthosis 10 about the parameters in the form of sensor values, sensor data, processed sensor data, or other data, for example statistical data or the like, are compared to the stored criteria. If the parameters or sensor data determined during the use correspond to a criterion, this is assessed as an event or time-limited activity. Such an event or such a time-limited activity is provided with a timestamp as a characteristic parameter or another characteristic parameter and stored in a memory of the control device 40 or a separate buffer memory. Multiple events or time-limited activities can have been previously defined, which are recognized and stored from a large number of combinations of determined parameters or additional information via the data provision device 70.

FIG. 2 shows a schematic representation of an orthopedic device 13 in the form of a prosthetic knee joint. The prosthetic knee joint 13 includes an upper part 11 and a lower part 12, which are rotatably mounted on one another around a pivot axis 14. The lower part 12 has connection means for fastening a prosthetic foot on its distal end. Multiple sensors 30 are also arranged on the lower part 12, in order to detect sensor data or parameters or information and state data. For example, spatial location information, axial forces, torques, angles, and/or accelerations can be measured via the sensors 30. The sensor values are transmitted to a control device 40, in which the devices necessary for processing the sensor signals are provided. These are in particular processors, storage devices, software, energy stores, and possibly transmitters and receivers for accepting and passing on signals. The processed sensor signals are used to control the resistance device 20, which is designed in the illustrated exemplary embodiment as a braking device, which influences a pivot of the upper part 11 relative to the lower part 12. In addition, a buffer memory 41 is arranged on the lower part 12, which communicates with the control device 40 and is equipped with an interface 42, via which the temporarily stored events and time-limited activities, parameters, or other data and information can be transmitted for external processing to an external data processing device 80. A display 43 can be arranged in the area of the pivot axis 14, in which, for example, a change of the load limiting values, a state of charge, or another information is displayed. An adjustment device 60 is assigned to the display 43, via which parameters of the parameters can be set. The display 41 can also be arranged at another point of the prosthetic knee joint or orthopedic device and can communicate wirelessly with the adjustment device 60, so that a setting can take place in a manner comfortably achievable for the user at any location, for example also via a mobile telephone, a tablet, or another graphic user interface.

FIG. 3 schematically shows the sequence of the method. First, events and/or time-limited activities and the characteristic parameters or parameter combinations to be attributed thereto are defined. Furthermore, criteria are defined which have to be met so that a certain curve of sensor data or a parameter or the presence of multiple sensor data or parameters at a certain point in time or over a certain period of time or in a certain chronological sequence qualify as a predefined event or a time-limited activity. These criteria or rules for the definition of events and/or time-limited activities are stored in the control device 40. The characteristic parameters to be added to the respective events or time-limited activities, which can also be determined via the sensors, are also predefined according to their type, wherein this definition is changeable. After these preparatory measures, information or data are transmitted via the sensors 30 or via other information sources 70, for example a data interface, via an interface 42 of the control device 40. If the orthopedic device is in the corresponding mode, in which an analysis of usage data is to take place, these sensor data, parameters, or information are analyzed and checked as to whether they match with the predefined criteria.

If it can be recognized on the basis of the detected sensor data, the data of the data interface or the data transmission device 70, and/or on the basis of other states that predefined events have taken place and/or time-limited activities have been carried out, the criteria for an event/an activity have been met. The corresponding event or the time-limited activity was recognized and is stored as a numeric value with a timestamp or another characteristic parameter or other characteristic parameters, for example in the form of data tuples.

The criteria to be met for the sensor data, parameters, or parameter combinations can be different for each event and/or each time-limited activity. The characteristic parameter or parameters to be added to the event and/or time-limited activity can also be different for each recognized event or each recognized time-limited activity. A fundamental delimitation of the signal values, maximum values, mean values, time intervals, or the like for the event is not provided. The sequence, the number of the entries, the length of the entries, and the meaning in the respective database are also freely selectable. The storage of usage data according to the above-described method has the advantage that an incremental storage of numeric values is possible. However, more detailed information about the actual progression of the load or usage of the orthopedic device are obtained from this number. The analysis of the usage data can also be carried out in dependence on various usage modes. In orthopedic devices of the lower extremity, for example, the steps can be counted and the execution of the steps can be monitored. An activation or deactivation of functions or special functions can be detected and chronologically assigned.

During walking, the occurrence of special loads can be detected. A simple detection of various usage situations follows, for example, in the assignment of a respective usage, which is of interest, in a table. A first field can characterize a step on the level, a second field the activation of a standing function, a third field a specific usage mode, a fourth field is reserved for the overload of the orthopedic device with respect to a specific load value, the fifth field identifies the function of sitting down, and the sixth field is provided for connecting a charging device.

Subcriteria can be provided for the respective fields, so that, for example, for the first field having the step on the level, parameters or features of the step can be detected, for example the step duration, the step length, the maximum knee angle in the swing phase, a maximum hydraulic force in the resistance device, the dissipated energy, the mean temperature of the hydraulic liquid, or an axial load, all provided with a timestamp, in order to further characterize the step. Not only instantaneous values at the point in time of a fulfilled criterion or a triggering event are stored here, but also information about a preceding progress, a sequence of measured values or parameters, or a period of time within which certain criteria have been met. These are, for example, extreme values, mean values, integral variables, or also nesting of events within a sequence of a movement, for example the number of the pedal revolutions during a continuous use of a bicycle mode.

The calculation and analysis of the parameters and additional information takes place during the use of the orthopedic device, in particular in the software of the control device 40. Alternatively, a separate component of the orthopedic device can be assigned, in which the hardware and software are present and to which the sensor values or other information are made available for analysis.

One advantage in relation to a continuous recording of raw data is the reduction of the storage requirement and the significant reduction of the data stream to be transmitted, wherein essential information about qualitative values, about the chronological distribution, and chronological and causative relationships can still be obtained from the stored data. A cumulative number of events or time-limited activities is stored via the number of the stored data tuples, the data analysis can thus be carried out in a significantly more detailed manner. For example, statistical load distributions, temperature curves, analyses of movement clusters, the chronological distribution and duration of activities and overloads can be reliably determined and analyzed. Technical designs are thus facilitated, since feedback can be obtained by everyday usage actions. In addition, adaptations to the respective user can be carried out more easily and questions with respect to the relevant user behavior can be answered. Due to the greatly compressed storage of information, it is possible to analyze the data directly within the system of the orthopedic device.

If the analysis has the result, for example, that the orthopedic device requires maintenance, such a report can be transmitted to the user, the orthopedic technician, or the producer via an interface or the interface. On the basis of the detected load cycles which can remain stored within the memory of the orthopedic device and the small amount of data, cumulative damage models can be used for calculating the maintenance requirement.

1. A method for analyzing usage data of an orthopedic device, having the following steps:
providing an orthopedic device (10, 13), which includes at least one sensor (30) for detecting sensor data and/or is connected to a data provision device (70) for transmitting sensor data, having at least one data processing device (40), which is coupled to the at least one sensor (30) and/or the data provision device (70) and in which at least one memory for storing and one processor for processing the sensor data are provided,
defining characteristic parameters and/or parameter combinations,
recognizing an event or a time-limited activity on the basis of the sensor data and/or a sensor data combination determined at a point in time or in a period of time,
determining parameters of the orthopedic device (10, 13) via the at least one sensor (30) and/or the data provision device (70) and associating the respective characteristic parameters and/or parameter combinations with an event or time-limited activities,
storing the occurrence of the event or the time-limited activity with at least one characteristic parameter or parameter combination.

2. The method as claimed in claim 1, characterized in that the occurrence of the respective event or the time-limited activity is stored with associated time information.

3. The method as claimed in claim 2, characterized in that the events or time-limited activities stored over a period of time are analyzed to determine loads over a usage period of time.

4. The method as claimed in claim 1, characterized in that multiple different sensors (30) are used to detect the parameters.

5. The method as claimed in claim 1, characterized in that the at least one sensor (30) or the sensors (30) is or are selected from a group of sensors which comprises force sensors, torque sensors, angle sensors, position sensors, velocity sensors, acceleration sensors, spatial location sensors, inertial measuring units, temperature sensors, pressure sensors, voltmeters, lead electrodes.

6. The method as claimed in claim 1, characterized in that, as a parameter which can trigger an event or can define a time-limited activity, at least one parameter range and/or one parameter limiting value is defined, the reaching and/or exceeding or falling below of which is defined as a condition for the occurrence of an event.

7. The method as claimed in claim 1, characterized in that discrete values, continuous values, and time variables are used as parameters.

8. The method as claimed in claim 1 any one of the preceding claims, characterized in that a prosthetic joint or an orthotic joint is used as the orthopedic device (10, 13).

9. The method as claimed in claim 1, characterized in that the type of the usage, the duration of the usage under certain conditions, and/or the reaching of a limiting value, in particular a load limit, is determined on the basis of the number and/or the chronological distribution of the respective stored events or time-limited activity.

10. The method as claimed in claim 1, characterized in that the events or time-limited activities are stored and analyzed in a control device (40) or transmitted to an external analysis device (80) for the analysis.

11. The method as claimed in claim 1, characterized in that the storage is deactivatable and/or takes place in dependence on the operating mode and/or a state of charge of an energy store.

12. The method as claimed in claim 1, characterized in that defining characteristic parameters and/or parameter combinations and associating the respective characteristic parameters and/or parameter combinations with an event or time-limited activities is parameterizable.

13. The method as claimed in claim 1, characterized in that the processing of parameters or an analysis takes place at predetermined times or in predetermined situations.
